(19) United States
(12) Patent Application Publication
(10) Pub. No.: US 2022/0240909 A1
(43) Pub. Date: Aug. 4, 2022

(54) CLAMP DEVICE FOR USE WITH A TOOL, SUCH AS A ROTATABLE MEDICAL TOOL

(71) Applicant: Proprio, Inc., Seattle, WA (US)

(72) Inventors: Jason A. Schoen, Seattle, WA (US); Stephen A. Morse, Woodinville, WA (US)

(21) Appl. No.: 17/162,888

(22) Filed: Jan. 29, 2021

(51) Int. Cl. A61B 17/00; A61B 34/20

(52) U.S. Cl. CPC A61B 17/00; A61B 34/20; A61B 17/16; A61B 2034/2072; A61B 2017/00477

(57) ABSTRACT

Clamp devices for use with rotatable medical tools and associated methods and systems are disclosed herein. In some embodiments, a clamp device includes a body having a first side portion and a second side portion. The clamp device can further include a first arm pivotably coupled to the first side portion of the body, and a second arm pivotably coupled to the second side portion of the body. The first arm can further include a first roller rotatably coupled to the first arm, and the second arm can further include a second roller rotatably coupled to the second arm. The first and second arms can be operably coupled to an actuation mechanism that is configured to pivot the first and second arms relative to the body of the clamp device to, for example, clamp a medical tool between the first and second rollers.

### TECHNICAL FIELD

The present technology generally relates to a clamp device, and more specifically, to a clamp device configured to be clamped to a rotatable medical tool.

### BACKGROUND

In a mediated reality system, an image processing system adds, subtracts, and/or modifies visual information representing an environment. For surgical applications, a mediated reality system may enable a surgeon to view a surgical site from a desired perspective together with contextual information that assists the surgeon in more efficiently and precisely performing surgical tasks. Such contextual information may include the position of objects within the scene, such as surgical tools. Specifically, the mediated reality system can include trackers configured to track markers or other identifiers fixed to objects of interest within the scene. While the objects of interest can be tracked when the markers are within view of the trackers, it can be difficult to track the objects when the markers are out of view of the trackers. For example, rotating a surgical tool can rotate the attached markers out of view of the trackers—thereby inhibiting the system from accurately tracking the position of the surgical tool.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic view of an imaging system in accordance with embodiments of the present technology.

FIGS. 2A and 2B are left and right isometric views, respectively, of a clamp device in a first position in accordance with embodiments of the present technology.

FIGS. 3A and 3B are left and right isometric views, respectively, of the clamp device of FIGS. 2A and 2B in a second position in accordance with embodiments of the present technology.

FIGS. 4A and 4B are an isometric view and a top view, respectively, of the clamp device of FIGS. 2A-3B in the first position in accordance with embodiments of the present technology and with a portion of the clamp device shown as transparent for clarity.

FIGS. 5A and 5B are isometric views of the clamp device of FIGS. 2A-4B rotatably secured to an object in accordance with embodiments of the present technology.

FIG. 6 is a flow diagram of a process or method for using a clamp device in accordance with embodiments of the present technology.

### DETAILED DESCRIPTION

Aspects of the present technology are directed generally to a clamp device configured to be clamped to a rotatable object, such as a rotatable medical tool. In several of the embodiments described below, for example, a clamp device includes (i) a body having a first side portion and a second side portion, (ii) a first arm pivotably coupled to the first side portion, and (iii) a second arm pivotably coupled to the second side portion. The clamp device can further include a first roller rotatably coupled to the first arm and a second roller rotatably coupled to the second arm. An actuation mechanism can be operably coupled to the first arm and the second arm and configured to pivot the first arm and the second arm relative to the body. For example, the actuation mechanism can pivot the first and second arms toward one another to clamp a rotatable medical tool against/between the first and second rollers. The rotatable tool can be a pedicle screw tap, a drill, or any other device that rotates or includes rotating elements.

In some aspects of the present technology, when the clamp device is clamped to an object, the clamp device is rotatably coupled to the object such that the object can freely rotate without changing an orientation of the clamp device. For example, when the object rotates, the first and second rollers can rotate against the object along parallel longitudinal axes such that the first and second arms remain in a generally fixed orientation relative to the object.

The clamp device can further include a plurality of markers (e.g., marker balls) releasably or integrally attached to the first arm, the second arm, or the body of the clamp device. The marker balls can be tracked by a mediated reality system and used to determine the position of an object or tool rotatably secured within the clamp device. In some aspects of the present technology, the object or tool can be rotated (e.g., during a surgical procedure) relative to the clamp device such that the markers remain visible to the mediated reality system while the object or tool is being used. Accordingly, the mediated reality system can continuously track the position of the object or tool.

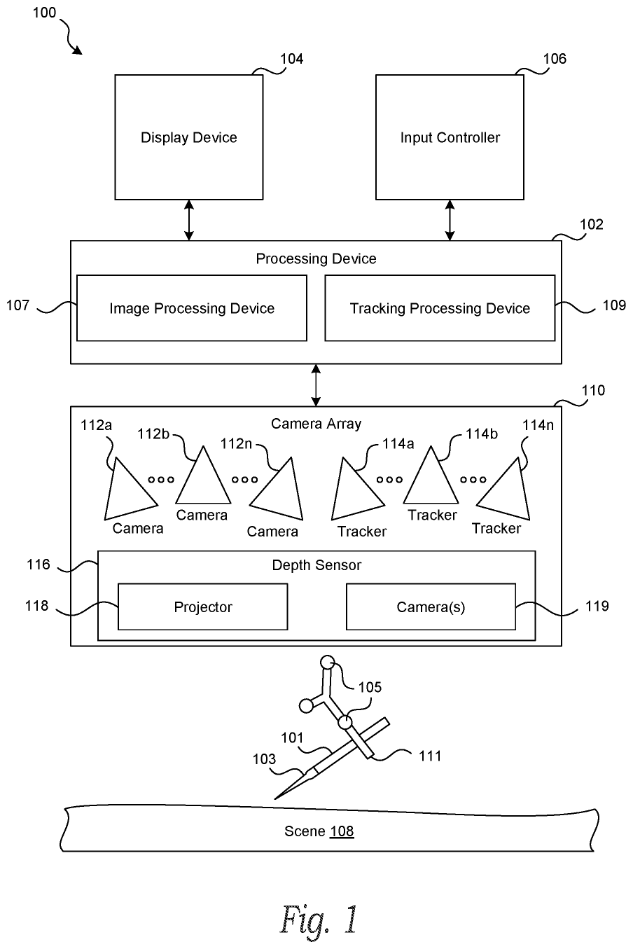

Specific details of several embodiments of the present technology are described herein with reference to FIGS. 1-6. The present technology, however, can be practiced without some of these specific details. In some instances, well-known structures and techniques often associated with clamps, rollers, pivots, fasteners, actuation mechanisms, camera arrays, light field cameras, image reconstruction, object tracking, etc., have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

The accompanying figures depict embodiments of the present technology and are not intended to be limiting of its scope. The sizes of various depicted elements are not necessarily drawn to scale, and these various elements can be arbitrarily enlarged to improve legibility. Component details can be abstracted in the figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

FIG. 1 is a schematic view of an imaging system 100 (“system 100”) in accordance with embodiments of the present technology. In some embodiments, the system 100 can be a synthetic augmented reality system, a mediated-reality imaging system, and/or a computational imaging system. In the illustrated embodiment, the system 100 includes a processing device 102 that is operably/communicatively coupled to one or more display devices 104, one or more input controllers 106, and a camera array 110. In other embodiments, the system 100 can comprise additional, fewer, or different components. In some embodiments, the system 100 can include some features that are generally similar or identical to those of the imaging systems disclosed in (i) U.S. patent application Ser. No. 16/586,375, titled “CAMERA ARRAY FOR A MEDIATED-REALITY SYSTEM,” and filed Sep. 27, 2019, and/or (ii) U.S. patent application Ser. No. 15/930,305, titled “METHODS AND SYSTEMS FOR IMAGING A SCENE, SUCH AS A MEDICAL SCENE, AND TRACKING OBJECTS WITHIN THE SCENE,” and filed May 12, 2020, each of which is incorporated herein by reference in its entirety.

In the illustrated embodiment, the camera array 110 includes a plurality of cameras 112 (identified individually as cameras 112a-112n) that are each configured to capture images of a scene 108 from a different perspective. The camera array 110 further includes a plurality of dedicated object trackers 114 (identified individually as trackers 114a-114n) configured to capture positional data of one more objects, such as a tool 101 (e.g., a surgical tool, a rotatable medical tool) having a tip 103, to track the movement and/or orientation of the objects through/in the scene 108. In some embodiments, the cameras 112 and the trackers 114 are positioned at fixed locations and orientations (e.g., poses) relative to one another. For example, the cameras 112 and the trackers 114 can be structurally secured by/to a mounting structure (e.g., a frame) at predefined fixed locations and orientations. In some embodiments, the cameras 112 can be positioned such that neighboring cameras 112 share overlapping views of the scene 108. Likewise, the trackers 114 can be positioned such that neighboring trackers 114 share overlapping views of the scene 108. Therefore, all or a subset of the cameras 112 and the trackers 114 can have different extrinsic parameters, such as position and orientation.

In some embodiments, the cameras 112 in the camera array 110 are synchronized to capture images of the scene 108 substantially simultaneously (e.g., within a threshold temporal error). In some embodiments, all or a subset of the cameras 112 can be light-field/plenoptic/RGB cameras that are configured to capture information about the light field emanating from the scene 108 (e.g., information about the intensity of light rays in the scene 108 and also information about a direction the light rays are traveling through space). Therefore, in some embodiments the images captured by the cameras 112 can encode depth information representing a surface geometry of the scene 108. In some embodiments, the cameras 112 are substantially identical. In other embodiments, the cameras 112 can include multiple cameras of different types. For example, different subsets of the cameras 112 can have different intrinsic parameters such as focal length, sensor type, optical components, and the like. The cameras 112 can have charge-coupled device (CCD) and/or complementary metal-oxide semiconductor (CMOS) image sensors and associated optics. Such optics can include a variety of configurations including lensed or bare individual image sensors in combination with larger macro lenses, micro-lens arrays, prisms, and/or negative lenses.

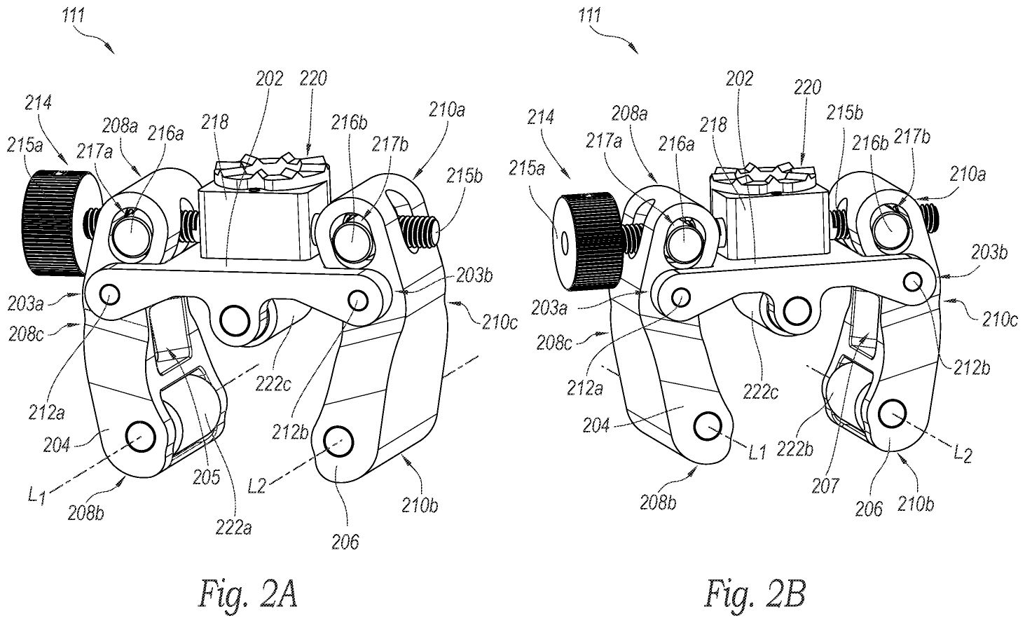

In some embodiments, the trackers 114 are imaging devices, such as infrared (IR) cameras that are each configured to capture images of the scene 108 from a different perspective compared to other ones of the trackers 114. Accordingly, the trackers 114 and the cameras 112 can have different spectral sensitives (e.g., infrared vs. visible wavelength). In some embodiments, the trackers 114 are configured to capture image data of a plurality of optical markers (e.g., fiducial markers, marker balls) in the scene 108, such as markers 105 coupled to the tool 101. In the illustrated embodiment, the markers 105 are attached to a clamp device 111 and secured to the tool 101 via the clamp device 111. As described in greater detail below with reference to FIGS. 2A-6, the clamp device 111 can be rotatably coupled to the tool 101. Accordingly, in some aspects of the present technology the tool 101 can be rotated relative to the clamp device 111 such that the markers 105 remain in view of the trackers 114 and do not become obscured even as the tool is rotated or otherwise moved during, for example, a surgical procedure. Thus, the system 100 can track the tool 101 during substantially the entire surgical procedure.

In the illustrated embodiment, the camera array 110 further includes a depth sensor 116. In some embodiments, the depth sensor 116 includes (i) one or more projectors 118 configured to project a structured light pattern onto/into the scene 108, and (ii) one or more cameras 119 (e.g., a pair of the cameras 119) configured to detect the structured light projected onto the scene 108 by the projector 118 to estimate a depth of a surface in the scene 108. The projector 118 and the cameras 119 can operate in the same wavelength and, in some embodiments, can operate in a wavelength different than the trackers 114 and/or the cameras 112. In other embodiments, the depth sensor 116 and/or the cameras 119 can be separate components that are not incorporated into an integrated depth sensor. In yet other embodiments, the depth sensor 116 can include other types of dedicated depth detection hardware such as a LiDAR detector, to estimate the surface geometry of the scene 108. In other embodiments, the camera array 110 can omit the projector 118 and/or the depth sensor 116.

In the illustrated embodiment, the processing device 102 includes an image processing device 107 (e.g., an image processor, an image processing module, an image processing unit) and a tracking processing device 109 (e.g., a tracking processor, a tracking processing module, a tracking processing unit). The image processing device 107 is configured to (i) receive images (e.g., light-field images, light field image data) captured by the cameras 112 of the camera array 110 and (ii) process the images to synthesize an output image corresponding to a selected virtual camera perspective. In the illustrated embodiment, the output image corresponds to an approximation of an image of the scene 108 that would be captured by a camera placed at an arbitrary position and orientation corresponding to the virtual camera perspective. In some embodiments, the image processing device 107 is further configured to receive depth information from the depth sensor 116 and/or calibration data to synthesize the output image based on the images, the depth information, and/or the calibration data. More specifically, the depth information and calibration data can be used/combined with the images from the cameras 112 to synthesize the output image as a 3D (or stereoscopic 2D) rendering of the scene 108 as viewed from the virtual camera perspective. In some embodiments, the image processing device 107 can synthesize the output image using any of the methods disclosed in U.S. patent application Ser. No. 16/457,780, titled “SYNTHESIZING AN IMAGE FROM A VIRTUAL PERSPECTIVE USING PIXELS FROM A PHYSICAL IMAGER ARRAY WEIGHTED BASED ON DEPTH ERROR SENSITIVITY,” filed Jun. 28, 2019, which is incorporated herein by reference in its entirety.

The image processing device 107 can synthesize the output image from images captured by a subset (e.g., two or more) of the cameras 112 in the camera array 110, and does not necessarily utilize images from all of the cameras 112. For example, for a given virtual camera perspective, the processing device 102 can select a stereoscopic pair of images from two of the cameras 112 that are positioned and oriented to most closely match the virtual camera perspective. In some embodiments, the image processing device 107 (and/or the depth sensor 116) is configured to estimate a depth for each surface point of the scene 108 relative to a common origin and to generate a point cloud and/or 3D mesh that represents the surface geometry of the scene 108. For example, in some embodiments the cameras 119 of the depth sensor 116 can detect the structured light projected onto the scene 108 by the projector 118 to estimate depth information of the scene 108. In some embodiments, the image processing device 107 can estimate depth from multiview image data from the cameras 112 using techniques such as light field correspondence, stereo block matching, photometric symmetry, correspondence, defocus, block matching, texture-assisted block matching, structured light, and the like, with or without utilizing information collected by the depth sensor 116. In other embodiments, depth may be acquired by a specialized set of the cameras 112 performing the aforementioned methods in another wavelength.

In some embodiments, the tracking processing device 109 can process positional data captured by the trackers 114 to track objects (e.g., the tool 101) within the vicinity of the scene 108. For example, the tracking processing device 109 can determine the position of the markers 105 in the 2D images captured by two or more of the trackers 114, and can compute the 3D position of the markers 105 via triangulation of the 2D positional data. More specifically, in some embodiments the trackers 114 include dedicated processing hardware for determining positional data from captured images, such as a centroid of the markers 105 in the captured images. The trackers 114 can then transmit the positional data to the tracking processing device 109 for determining the 3D position of the markers 105. In other embodiments, the tracking processing device 109 can receive the raw image data from the trackers 114. In a surgical application, for example, the tracked object may comprise a surgical instrument, a hand or arm of a physician or assistant, and/or another object having the markers 105 mounted thereto. In some embodiments, the processing device 102 may recognize the tracked object as being separate from the scene 108, and can apply a visual effect to distinguish the tracked object such as, for example, highlighting the object, labeling the object, or applying a transparency to the object.

In some embodiments, functions attributed to the processing device 102, the image processing device 107, and/or the tracking processing device 109 can be practically implemented by two or more physical devices. For example, in some embodiments a synchronization controller (not shown) controls images displayed by the projector 118 and sends synchronization signals to the cameras 112 to ensure synchronization between the cameras 112 and the projector 118 to enable fast, multi-frame, multi-camera structured light scans. Additionally, such a synchronization controller can operate as a parameter server that stores hardware specific configurations such as parameters of the structured light scan, camera settings, and camera calibration data specific to the camera configuration of the camera array 110. The synchronization controller can be implemented in a separate physical device from a display controller that controls the display device 104, or the devices can be integrated together.

The processing device 102 can comprise a processor and a non-transitory computer-readable storage medium that stores instructions that when executed by the processor, carry out the functions attributed to the processing device 102 as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

The virtual camera perspective can be controlled by an input controller 106 that provides a control input corresponding to the location and orientation of the virtual camera perspective. The output images corresponding to the virtual camera perspective are outputted to the display device 104. The display device 104 is configured to receive the output images (e.g., the synthesized three-dimensional rendering of the scene 108) and to display the output images for viewing by one or more viewers. The processing device 102 can process received inputs from the input controller 106 and process the captured images from the camera array 110 to generate output images corresponding to the virtual perspective in substantially real-time as perceived by a viewer of the display device 104 (e.g., at least as fast as the framerate of the camera array 110). Additionally, the display device 104 can display a graphical representation of any tracked objects within the scene 108 (e.g., the tool 101) on/in the image of the virtual perspective.

The display device 104 can comprise, for example, a head-mounted display device, a monitor, a computer display, and/or another display device. In some embodiments, the input controller 106 and the display device 104 are integrated into a head-mounted display device and the input controller 106 comprises a motion sensor that detects position and orientation of the head-mounted display device. The virtual camera perspective can then be derived to correspond to the position and orientation of the head-mounted display device 104 in the same reference frame and at the calculated depth (e.g., as calculated by the depth sensor 116) such that the virtual perspective corresponds to a perspective that would be seen by a viewer wearing the head-mounted display device 104. Thus, in such embodiments the head-mounted display device 104 can provide a real-time rendering of the scene 108 as it would be seen by an observer without the head-mounted display device 104. Alternatively, the input controller 106 can comprise a user-controlled control device (e.g., a mouse, pointing device, handheld controller, gesture recognition controller) that enables a viewer to manually control the virtual perspective displayed by the display device 104.

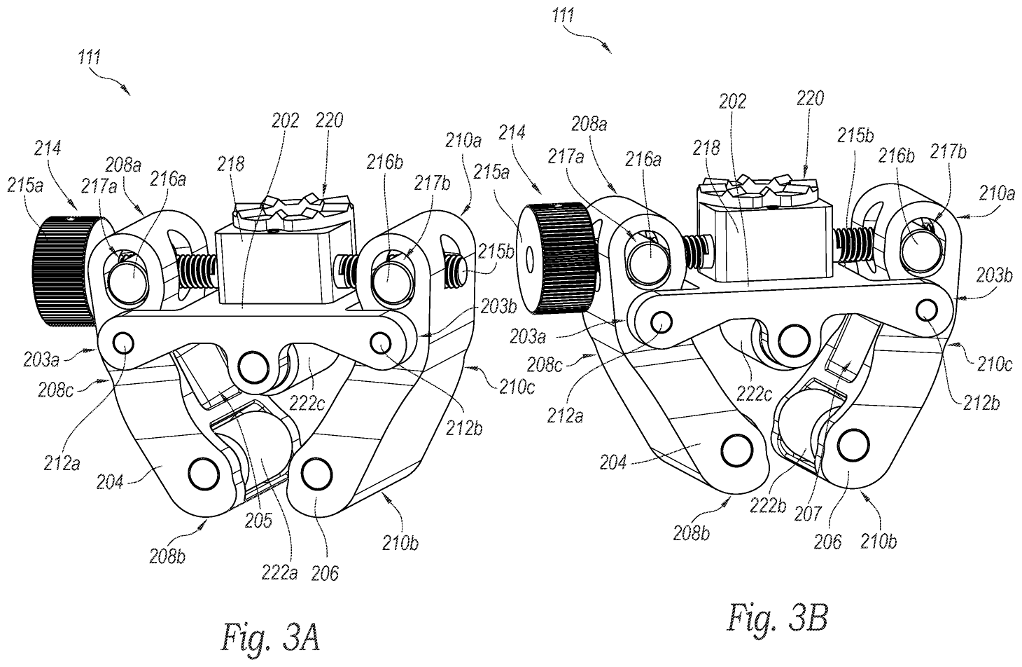

FIGS. 2A and 2B are left and right isometric views, respectively, of the clamp device 111 of FIG. 1 in a first position (e.g., an open position, an unclamped position, a widened position) in accordance with embodiments of the present technology. FIGS. 3A and 3B are left and right isometric views, respectively, of the clamp device 111 in a second position (e.g., a closed position, a clamped position, a narrowed position) in accordance with embodiments of the present technology. Referring to FIGS. 2A-3B together, in the illustrated embodiment the clamp device 111 includes (i) a body 202, (ii) a first arm 204 coupled to the body 202, (iii) a second arm 206 coupled to the body 202, and (iv) an actuation mechanism 214 operably coupled to the first and second arms 204, 206. The actuation mechanism 214 is operable (e.g., actuatable, rotatable) to move the first and second arms 204, 206 between the first and second positions to, for example, clamp a medical tool therebetween.

More specifically, in the illustrated embodiment the body 202 has a first side portion 203a and a second side portion 203b. The first arm 204 is pivotably coupled to the first side portion 203a via a first pivot member 212a (e.g., a rod, elongate member), and the second arm 206 is pivotably coupled to the second side portion 203b via a second pivot member 212b. The first arm 204 has a first end portion 208a, a second end portion 208b opposite the first end portion 208a, and a middle portion 208c extending between the first and second end portions 208a-b. Similarly, the second arm has a first end portion 210a, a second end portion 210b opposite the first end portion 210a, and a middle portion 210c extending between the first and second end portions 210a-b. In the illustrated embodiment, the first pivot member 212a extends through and pivotably couples the middle portion 208c of the first arm 204 to the first side portion 203a of the body 202. Likewise, the second pivot member 212b extends through and pivotably couples the middle portion 210c of the second arm 206 to the second side portion 203b of the body 202. The first arm 204 and the second arm 206 can have different, generally similar, substantially identical, or the same dimensions. In some embodiments, the first arm and the second arm 206 are identical. The body 202, the first arm 204, and the second arm 206 can be made of the same or different materials, such as metals, composite materials, and/or other suitably strong and rigid materials.

In the illustrated embodiment, the first arm 204 further includes a first arm cavity 205 positioned near the middle portion 208c of the first arm 204, and the second arm 206 further includes a second arm cavity 207 positioned near the middle portion 210c of the second arm 206. Including the first arm cavity 205 and/or the second arm cavity 207 can reduce the weight and/or manufacturing cost of the clamp device 111. The dimensions of the first arm cavity 205 and the second arm cavity 207 can be the same or different and, in some embodiments, the first arm cavity 205 and/or the second arm cavity 207 can be omitted.

The clamp device 111 can further include a plurality of rollers 222 (identified individually as first through third rollers 222a-222c, respectively). In the illustrated embodiment, (i) the first roller 222a is carried by and rotatably coupled to the second end portion 208b of first arm 204, (ii) the second roller 222b is carried by and rotatably coupled to the second end portion 210b of the second arm 206, and (iii) the third roller 222c is carried by and rotatably coupled to the body 202. Each of the rollers 222 can be configured to rotate about respective and generally parallel longitudinal axes defined by a central axis of each of the rollers 222. For example, in the illustrated embodiment the first roller 222a is configured to rotate about a first longitudinal axis L₁ (FIGS. 2A and 2B) and the second roller 222b is configured to rotate about a second longitudinal axis L₂ (FIGS. 2A and 2B) parallel to the first longitudinal axis L₁. In other embodiments, the clamp device 111 can include more or fewer of the rollers 222 and/or the rollers 222 can be positioned differently. For example, the first arm 204 and/or the second arm 206 can include more than one roller (e.g., including one or more additional rollers proximate to the middle portion 208c and/or the middle portion 210c), the third roller 222c can be omitted, and so on. The rollers 222 can be made of Delrin, or other suitable metals, composites, and the like.

In some embodiments, the clamp device 111 can additionally include a coupling member 218 coupled to or integrally formed with the body 202. The coupling member 218 can include an attachment portion 220 for releasably or integrally attaching one or more markers or other objects (not shown) to the clamp device 111. For example, the attachment portion 220 can be used to releasably attach a rigid constellation of markers—such as the markers 105 of FIG. 1—to the clamp device 111. The attachment portion 220 can comprise a friction-fitting socket, a ball socket, screw threading, clamp, adhesive, or other means for releasably or integrally attaching the markers to the clamp device 111.

The actuation mechanism 214 is operably coupled to the first and second arms 204, 206 and configured to move (e.g., pivot, rotate) the first and second arms 204, 206 relative to the body 202. In the illustrated embodiment, for example, the actuation mechanism 214 is a screw having a head 215a and a threaded body 215b extending from the head 215a. In some embodiments, the threaded body 215b is threadably coupled to a first arm coupling 216a and a second arm coupling 216b. The first arm coupling 216a is coupled to the first end portion 208a of first arm 204, and the second arm coupling 216b is coupled to the first end portion 210a of second arm 206. More specifically, for example, the first arm coupling 216a can be a barrel nut or other threaded fastener secured in a first opening 217a in the first end portion 208a and the second arm coupling 216b can be a barrel nut or other threaded fastener secured in a second opening 217b in the first end portion 210a. In other embodiments, the actuation mechanism 214 can be directly coupled to the first and second arms 204, 206 via, for example, threaded openings extending therethrough.

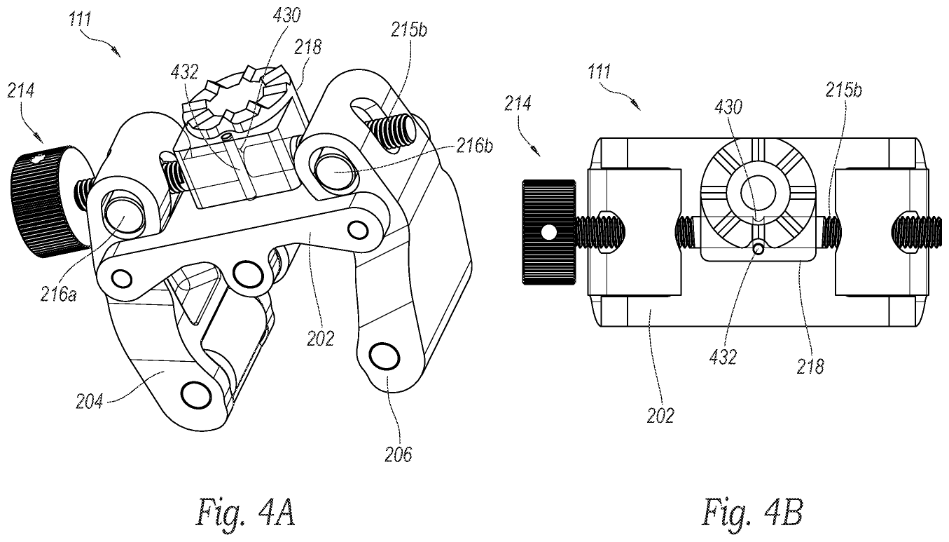

In the illustrated embodiment, the threaded body 215b of the actuation mechanism 214 extends through the coupling member 218 (e.g., between the first and second arm couplings 216a-b). FIGS. 4A and 4B are an isometric view and a top view, respectively, of the clamp device 111 in the first position and showing the coupling member 218 as transparent for clarity. Referring to FIGS. 4A and 4B together, in some embodiments the threaded body 215b can have a groove 430 extending at least partially about a circumference thereof. In some embodiments, the threaded body 215b is only threaded along a portion extending through the first and second arm couplings 216a-b. In the illustrated embodiment, the clamp device 111 (e.g., the coupling member 218) further includes a post 432 (e.g., an elongate member, restraint, pin) configured to engage the groove 430. That is, the post 432 can have a size (e.g., diameter, length) selected to correspond to a size of the groove 430 and can be positioned at least partially within the groove 430. The engagement of the post 432 within the groove 430 inhibits the actuation mechanism 214 from moving linearly (e.g., relative to the body 202) when the actuation mechanism 214 is actuated. That is, the post 432 acts to lock the position of the actuation mechanism 214 such that movement (e.g., rotation) of the actuation mechanism 214 is transferred to movement of the first and second arms 204, 206 rather than linear (or other undesired) motion of the actuation mechanism 214.

Referring again to FIGS. 2A-3B together, the actuation mechanism 214 can be actuated to drive the clamp device 111 between the first and second positions (e.g., from the first position to the second position and/or from the second position to the first position). In some embodiments, for example, a user can grip and rotate the head 215a of the actuation mechanism 214 to rotate the threaded body 215b relative to the first and second arm couplings 216a-b. Because the first and second arm couplings 216a-b are fixedly coupled to the first and second arms 204, 206, respectively, the rotation of the threaded body 215b causes the first and second arm couplings 216a-b to translate along the length of the threaded body 215b toward or away from one another.

For example, rotating the actuation mechanism 214 in a first direction (e.g., clockwise direction) can move the first and second arm couplings 216a-b away from one another (e.g., and away from the coupling member 218) to increase the distance between the first end portion 208a of the first arm 204 and the second end portion 210b of the second arm 206. The movement of the first end portions 208a, 210a away from one another causes the first and second arms 204, 206 to pivot about the first and second pivot members 212a-b, respectively, to cause the second end portions 208b, 210b to move toward one another from, for example, the first position shown in FIGS. 2A and 2B to the second position shown in FIGS. 3A and 3B. This movement of the first and second arms 204, 206 further acts to move the first and second rollers 222a-b (and their respective rotational axes L₁ and L₂) toward one another and the third roller 222c.

Conversely, rotating the actuation mechanism 214 in a second direction (e.g., counterclockwise direction) can move the first and second arm couplings 216a-b toward one another (e.g., toward the coupling member 218) to decrease the distance between the first end portion 208a of the first arm 204 and the first end portion 210a of the second arm 206. The movement of the first end portions 208a, 210a toward one another causes the first and second arms 204, 206 to pivot about the first and second pivot members 212a-b, respectively, to cause the second end portions 208b, 210b to move away from one another from, for example, the second position shown in FIGS. 3A and 3B to the first position shown in FIGS. 2A and 2B. This movement of the first and second arms 204, 206 further acts to move the first and second rollers 222a-b (and their respective rotational axes L₁ and L₂) away from one another and the third roller 222c.

In some aspects of the present technology, the first and second arm couplings 216a-b and the first and second arms 204, 206 can be sized, oriented, and or otherwise configured such that actuation of the actuation mechanism 214 pivots the first and second arms 204, 206 in coordination/synchronization. For example, in some embodiments rotation of the actuation mechanism 214 pivots the first arm 204 and the second arm 206 through substantially equal angles and/or along paths of substantially equal distance. Moreover, with additional reference to FIGS. 4A and 4B, the engagement of the post 432 with the groove 430 ensures that the actuation mechanism 214 remains in a constant (e.g., centered) position relative to the body 202 and helps ensure that the rotary motion of the actuation mechanism 214 is translated to linear motion of the first and second arm couplings 216a-b. In additional aspects of the present technology, this coordinated motion of the first arm 204 and the second arm 206 can allow the clamp device 111 to automatically center on an object placed between the first arm 204 and the second arm 206.

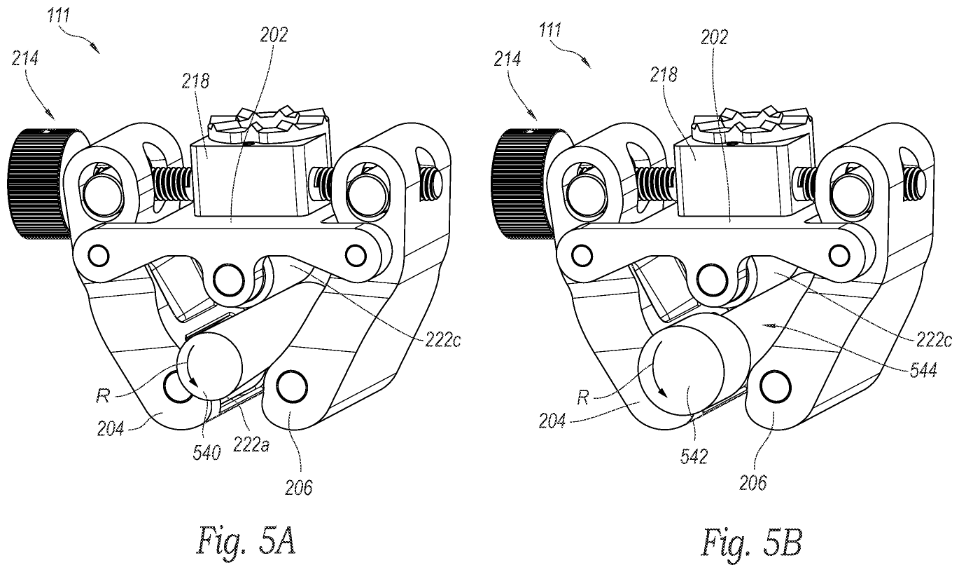

FIG. 5A is an isometric view of the clamp device 111 secured to an object 540 in accordance with embodiments of the present technology. The object 540 can be a surgical tool having a substantially circular radial cross section, such as a biopsy punch, a syringe, a catheter delivery system, a stent delivery system, a stylet, a probe, and the like. In some embodiments, the object 540 can be a surgical instrument including a rotating element, such as a pedicle screw tap or a drill. In the illustrated embodiment, the clamp device 111 has been moved from the first position (FIGS. 2A and 2B) to the second position (FIGS. 3A and 3B) to secure the clamp device 111 to the object 540. More specifically, with additional reference to FIGS. 2A-3B, the clamp device 111 can be positioned around the object 540 in the first position and then the actuation mechanism 214 can be actuated to move the clamp device 111 to the second position in which the rollers 222 (partially obscured in FIG. 5A) contact an outer surface of the object 540.

When the clamp device 111 is secured to the object 540, the rollers 222 are the only portions of the clamp device 111 that contact the object 540 such that the clamp device 111 and the object 540 can rotate relative to one another. For example, the object 540 can rotate freely (e.g., in the direction of arrow R) against the rollers 222 while the body 202 and the first and second arms 204, 206 of the clamp device 111 remain substantially stationary. Further, in some embodiments the positioning of the rollers 222 and the coordinated/synchronized movement of the first and second arms 204, 206 can cause the clamp device 111 to automatically center about the object 540 such that, for example, the object 540 is secured at a fixed distance and orientation relative to the coupling member 218. In some embodiments, the engagement (e.g., frictional forces) between the rollers 222 and the object 540 can inhibit or even prevent axial movement of the object 540 relative to the clamp device 111. For example, the actuation mechanism 214 can be actuated to tightly clamp the rollers 222 about the object 540 to inhibit axial movement thereof.

In other embodiments, the object and/or the rollers 222 can include additional features that inhibit the object from moving axially relative to the clamp device 111. For example, FIG. 5B is an isometric view of the clamp device 111 secured to an object 542 in accordance with embodiments of the present technology. The object 542 can include a contact region 544 configured to receive the rollers 222 and inhibit the object 542 from moving axially relative to the clamp device 111. In the illustrated embodiment, for example, the contact region 544 is a groove extending about the circumference of object 542, and having an axial length equal to or greater than an axial length of the rollers 222, and a depth sufficient to (i) inhibit the object 542 from moving axially relative to the clamp device 111 while (ii) still permitting the clamp device 111 to rotate freely relative to the object 540. That is, the contact region 544 can act as a track that maintains the position of the rollers 222 along the object 542. In other embodiments, the contact region 544 can be a patterned surface, and the rollers 222 can include a corresponding patterned surface such that the rollers 222 and contact region 544 interface and inhibit the clamp device 111 and the object 540 from moving axially relative to each other. For example, the contact region 544 can have a plurality of protrusions and/or recess, and the rollers 222 can be configured with a corresponding plurality of protrusions and/or recess such that the rollers 222 and contact region 544 interface and inhibit the clamp device 111 and the object 540 from moving axially relative to one another.

Referring to FIGS. 1-5B together, in some aspects of the present technology the rotatable coupling of the clamp device 111 to the tool 101 (or the object 540, the object 542, etc.) can allow the markers 105 attached thereto to remain in view of the camera array 110 when the tool 101 rotates. For example, a user can hold the clamp device 111 while the tool 101 rotates such that the markers 105 remain substantially stationary and in view of the trackers 114 as the tool 101 rotates. Accordingly, the trackers 114 can continuously image the markers 105 to track the position of the tool 101. In some embodiments, the rotatable aspect of the clamp device 111 can inhibit the markers 105 from being obscured by other instruments, tools, the appendages of a user, or other such objects.

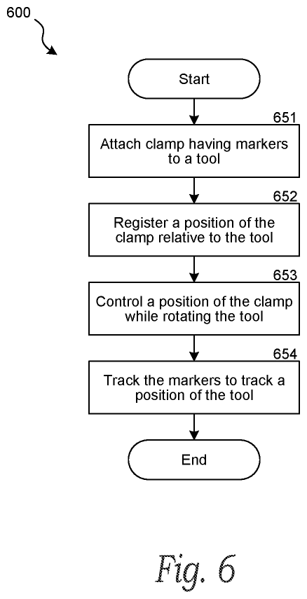

FIG. 6 is a flow diagram of a process or method for using the clamp device 111 in accordance with embodiments of the present technology. Although some features of the method 650 are described in the context of the embodiments shown in FIGS. 1-5B for the sake of illustration, one skilled in the art will readily understand that the method 650 can be carried out using other suitable systems and/or device described herein. Furthermore, while the method 650 is described in the context of the rotatable medical tool 101, the method 650 and the clamp device 111 can be utilized with other tools and/or objects.

At block 651, the method 650 includes rotatably attaching the clamp device 111 with the markers 105 to the tool 101. For example, as described in detail above a user can actuate the actuation mechanism 214 to move the clamp device 111 from the first position (FIGS. 2A and 2B) to the second position (FIGS. 3A and 3B) to move the first and second arms 204, 206 inward and bring the rollers 222 into contact with the tool 101. In some embodiments, where the tool 101 includes a specified contact region (e.g., the contact region 544 shown in FIG. 5B), the method 650 can include bringing the rollers 222 into contact with the contact region (e.g., positioning the rollers 222 in a track along the tool 1010). The markers 105 can be attached to the coupling portion 218 of the clamp device 111 before or after the clamp device 111 is secured to the tool 101.

At block 652, the method 650 includes registering/calibrating a position and/or orientation of the clamp device 111 relative to the tool 101. For example, the system 100 or a dedicated registration device can determine a fixed position and/or a fixed orientation of the clamp device 111 and/or the markers 105 relative to the tool 101 such that, after calibration, the system 100 can determine the position of the tool 101 (e.g., a position of the tip 103 of the tool 101) in the scene 108 based on a tracked position of the markers 105.

At block 653, the method 650 includes controlling a position of the clamp device 111 relative to the camera array 110 while rotating the tool 101 relative to the clamp device 111. For example, the user can maintain the position of the clamp device 111 (e.g., by grasping the clamp device 111) such that the markers 105 remain in view of the camera array 110 while the tool 101 rotates freely. More specifically, the tool 101 can be a surgical drill or other instrument that is rotated during a surgical procedure. Either the surgeon or another user (e.g., an assistant) can maintain the position of the clamp device 111 as the tool 101 is rotated during the procedure.

At block 654, the method 650 includes tracking the markers 105 with the camera array 110 (e.g., the trackers 114) to track the position of the tool 101 within the scene 108. \For example, the system 100 can determine the position of the tool 101 within the scene 108 based on the tracked location of the markers 105 and the determined registration/calibration (block 652) of the position of the markers 105 relative to the tool 101. In some embodiments, the tracked position of the tool 101 can be displayed on the display device 104.

The following examples are illustrative of several embodiments of the present technology:
1. A clamp device configured to be clamped to a rotatable medical tool, the clamp device comprising:
a body having a first side portion and a second side portion;
a first arm pivotably coupled to the first side portion of the body;
a second arm pivotably coupled to the second side portion of the body;
a first roller rotatably coupled to the first arm;
a second roller rotatably coupled to the second arm; and
an actuation mechanism operably coupled to the first arm and the second arm, wherein the actuation mechanism is configured to pivot the first arm and the second arm relative to the body.

2. The clamp device of example 1 wherein the actuation mechanism is actuatable to pivot the first arm toward the second arm to move the first roller toward the second roller.

3. The clamp device of example 1 or example 2 wherein the first roller is aligned along and configured to rotate about a first longitudinal axis, wherein the second roller is aligned along and configured to rotate about a second longitudinal axis, wherein the first longitudinal axis is generally parallel to the second longitudinal axis, and wherein the actuation mechanism is configured to move the first arm and the second arm relative to the body in a direction generally orthogonal to the first and second longitudinal axes.

4. The clamp device of any one of examples 1-3 wherein actuation of the actuation mechanism simultaneously pivots the first arm and the second arm through a same angle relative to the body.

5. The clamp device of any one of examples 1˜4 wherein the first arm and the second arm are substantially identical, wherein the first and second arms each include (a) a first end portion, (b) a second end portion, and (c) a middle portion extending between the first and second end portions, wherein the middle portions of the first and second arms are pivotably coupled to the body, wherein the actuation mechanism is operably coupled to the first end portions of the first and second arms, wherein the first roller is rotatably mounted to the second end portion of the first arm, and wherein the second roller is rotatable mounted to the first end portion of the first arm.

6. The clamp device of example 5, further comprising a marker-ball connector coupled to the body between the first end portion of the first arm and the first end portion of the second arm.

7. The clamp device of any one of examples 1-6, further comprising a third roller carried by the body, wherein the actuation mechanism is actuatable in (a) a first direction to pivot the first and second arms toward the third roller and (b) in a second direction to pivot the first and second arms away from the third roller.

8. The clamp device of example 7 wherein actuating the actuation mechanism in the first direction centers the rotatable medical tool between the first roller, the second roller, and the third roller.

9. The clamp device of any one of examples 1-8, further comprising a pin coupled to the body, wherein the pin is positioned to engage a portion of the actuation mechanism to inhibit linear movement of the actuation mechanism.

10. The clamp device of example 9 wherein the portion of the actuation mechanism is a circumferential groove.

11. A clamp device, comprising:
a body, having a first side and a second side;
a first arm operably coupled to the first side of the body by a first pivot;
a second arm operably coupled to the second side of the body by a second pivot; a first roller operably coupled to an end portion of the first arm;
a second roller operably coupled to an end portion of the second arm;
a third roller operably coupled to the body of the clamp device; and
an actuation mechanism operably coupled to the first arm and the second arm, wherein the actuation mechanism is actuatable to pivot the first roller about the first pivot and to pivot the second roller about the second pivot.

12. The clamp device of example 11 wherein the first roller, the second roller, and the third roller are each configured to rotate about a longitudinal axis, and wherein the longitudinal axes are substantially parallel to one another.

13. The clamp device of example 11 or example 12 wherein the end portion of the first arm is a first end portion, wherein the first arm further includes a second end portion opposite the first end portion, wherein the end portion of the second arm is a first end portion, wherein the second arm further includes a second end portion opposite the first end portion, and wherein the actuation mechanism is coupled to the second end portion of the first arm and the second end portion of the second arm.

14. The clamp device of example 13, further comprising a marker-ball connector coupled to the body between the second end portion of the first arm and the second end portion of the second arm.

15. The clamp device of any one of examples 11-14 wherein the actuation mechanism is actuatable (a) in a first direction to pivot the first roller and the second roller toward one another and (b) in a second direction to pivot the first roller and the second roller away from one another.

16. The clamp device of any one of examples 11-15 wherein actuation of the actuation mechanism is configured to pivot the first roller and the second roller through substantially equal angles.

17. A method for tracking a tool, the method comprising:
positioning a clamp device about the tool;
actuating the clamp device to bring a plurality of rollers of the clamp device into contact with the tool;
determining a fixed position and orientation of the tool relative to a plurality of markers coupled to the clamp device;
rotating the tool relative to the clamp device;
tracking a position of the markers as the tool rotates; and determining a position of the tool based on the position of the markers and the determined fixed position and orientation of the tool relative to the markers.

18. The method of example 17 wherein determining the position of the tool includes determining the position of a tip of the tool.

19. The method of example 17 or example 18 wherein the tool is a surgical tool.

20. The method of any one of examples 17-19 wherein tracking the position of the markers includes tracking the position with a plurality of trackers, and wherein rotating the tool relative to the clamp device includes maintaining an orientation of the clamp device relative to at least some of the trackers.

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A clamp device configured to be clamped to a rotatable medical tool, the clamp device comprising:
a body having a first side portion and a second side portion;
a first arm pivotably coupled to the first side portion of the body;
a second arm pivotably coupled to the second side portion of the body;
a first roller rotatably coupled to the first arm;
a second roller rotatably coupled to the second arm; and
an actuation mechanism operably coupled to the first arm and the second arm, wherein the actuation mechanism is configured to pivot the first arm and the second arm relative to the body.

2. The clamp device of claim 1 wherein the actuation mechanism is actuatable to pivot the first arm toward the second arm to move the first roller toward the second roller.

3. The clamp device of claim 1 wherein the first roller is aligned along and configured to rotate about a first longitudinal axis, wherein the second roller is aligned along and configured to rotate about a second longitudinal axis, wherein the first longitudinal axis is generally parallel to the second longitudinal axis, and wherein the actuation mechanism is configured to move the first arm and the second arm relative to the body in a direction generally orthogonal to the first and second longitudinal axes.

4. The clamp device of claim 1 wherein actuation of the actuation mechanism simultaneously pivots the first arm and the second arm through a same angle relative to the body.

5. The clamp device of claim 1 wherein the first arm and the second arm are substantially identical, wherein the first and second arms each include (a) a first end portion, (b) a second end portion, and (c) a middle portion extending between the first and second end portions, wherein the middle portions of the first and second arms are pivotably coupled to the body, wherein the actuation mechanism is operably coupled to the first end portions of the first and second arms, wherein the first roller is rotatably mounted to the second end portion of the first arm, and wherein the second roller is rotatable mounted to the first end portion of the first arm.

6. The clamp device of claim 5, further comprising a marker-ball connector coupled to the body between the first end portion of the first arm and the first end portion of the second arm.

7. The clamp device of claim 1, further comprising a third roller carried by the body, wherein the actuation mechanism is actuatable in (a) a first direction to pivot the first and second arms toward the third roller and (b) in a second direction to pivot the first and second arms away from the third roller.

8. The clamp device of claim 7 wherein actuating the actuation mechanism in the first direction centers the rotatable medical tool between the first roller, the second roller, and the third roller.

9. The clamp device of claim 1, further comprising a pin coupled to the body, wherein the pin is positioned to engage a portion of the actuation mechanism to inhibit linear movement of the actuation mechanism.

10. The clamp device of claim 9 wherein the portion of the actuation mechanism is a circumferential groove.

11. A clamp device, comprising:
a body, having a first side and a second side;
a first arm operably coupled to the first side of the body by a first pivot;
a second arm operably coupled to the second side of the body by a second pivot;
a first roller operably coupled to an end portion of the first arm;
a second roller operably coupled to an end portion of the second arm;
a third roller operably coupled to the body of the clamp device; and
an actuation mechanism operably coupled to the first arm and the second arm, wherein the actuation mechanism is actuatable to pivot the first roller about the first pivot and to pivot the second roller about the second pivot.

12. The clamp device of claim 11 wherein the first roller, the second roller, and the third roller are each configured to rotate about a longitudinal axis, and wherein the longitudinal axes are substantially parallel to one another.

13. The clamp device of claim 11 wherein the end portion of the first arm is a first end portion, wherein the first arm further includes a second end portion opposite the first end portion, wherein the end portion of the second arm is a first end portion, wherein the second arm further includes a second end portion opposite the first end portion, and wherein the actuation mechanism is coupled to the second end portion of the first arm and the second end portion of the second arm.

14. The clamp device of claim 13, further comprising a marker-ball connector coupled to the body between the second end portion of the first arm and the second end portion of the second arm.

15. The clamp device of claim 11 wherein the actuation mechanism is actuatable (a) in a first direction to pivot the first roller and the second roller toward one another and (b) in a second direction to pivot the first roller and the second roller away from one another.

16. The clamp device of claim 11 wherein actuation of the actuation mechanism is configured to pivot the first roller and the second roller through substantially equal angles.

17. A method for tracking a tool, the method comprising:
positioning a clamp device about the tool;
actuating the clamp device to bring a plurality of rollers of the clamp device into contact with the tool;
determining a fixed position and orientation of the tool relative to a plurality of markers coupled to the clamp device;
rotating the tool relative to the clamp device;
tracking a position of the markers as the tool rotates; and
determining a position of the tool based on the position of the markers and the determined fixed position and orientation of the tool relative to the markers.

18. The method of claim 17 wherein determining the position of the tool includes determining the position of a tip of the tool.

19. The method of claim 17 wherein the tool is a surgical tool.

20. The method of claim 17 wherein tracking the position of the markers includes tracking the position with a plurality of trackers, and wherein rotating the tool relative to the clamp device includes maintaining an orientation of the clamp device relative to at least some of the trackers.
